(19) United States
(12) Patent Application Publication
(10) Pub. No.: US 2020/0368546 A1
(43) Pub. Date: Nov. 26, 2020

(54) Method of Detection and Measurement of a Life Force Energy, Also Known as KELEA in Humans, Animals, Plants, Equipment, and the Environment

(71) Applicant: William John Martin, South Pasadena, CA (US)

(72) Inventor: William John Martin, South Pasadena, CA (US)

(21) Appl. No.: 16/421,344

(22) Filed: May 23, 2019

(51) Int. Cl. A61N 5/00; A61J 3/00

(52) U.S. Cl. CPC A61N 5/00; A61J 3/00

(57) ABSTRACT

An assay procedure is described, which allows for the assessment in humans and in animals of a life force energy, which the Applicant refers to as KELEA, being an abbreviation for kinetic energy limiting electrostatic attraction KELEA serves as a non-food calorie source of cellular energy in what the Applicant calls the alternative cellular energy (ACE) pathway. A deficiency in the ACE pathway can contribute to various illnesses. Conversely, an adequate ACE pathway can assist in the healing process. The measurement of KELEA is based on the earlier finding that it can add to the measured weight of dipolar materials, including those containing cellulose. This later grouping extends to paper. KELEA can be transferred to, or alternatively, withdrawn from, paper, as determined by an increase or a decrease, respectively, in the measured weight of a sheet of paper. An increase or a decrease in the weight of a sheet of paper held by an individual provides an indication of adequacy or deficiency of the ACE pathway in the individual. Using the assay, procedures can be undertaken, including exposure to KELEA activated water and modifying mental attitudes, to convert a KELEA withdrawing result to a KELEA contributing result. The assay should find widespread use in advancing an understanding of the ACE pathway and its importance in human and animal wellness

### RELATED PUBLICATION BY THE APPLICANT

1. Martin, W. J. (2003) Stealth Virus Culture Pigments, A Potential Source of Cellular Energy. Experimental Molecular Pathology, 74, 210-223.
2. Martin, W. J. (2005) Alternative Cellular Energy Pigments from Bacteria of Stealth Virus Infected Individuals. Experimental Molecular Pathology, 78, 217-217.
3. Martin W J (2014). Stealth Adapted Viruses; Alternative Cellular Energy (ACE) & KELEA Activated Water. Author House, IN. pp 321. ISBN 978-1-4969-0496-6.
4. Martin, W. J. (2014) KELEA Activated Water—Enhancing the Alternative Cellular Energy (ACE) Pathway. In Stealth Adapted Viruses; Alternative Cellular Energy (ACE) & KELEA Activated Water. Author House, Bloomington, Ind., USA, pp. 115-144.
5. Martin, W. J. (2014) KELEA Activated Water Leading to Improved Quantity & Quality of Agricultural Crops. Advances in Plants & Agriculture Research, 2, 00033.
6. Martin, W. J. (2015) Therapeutic Potential of KELEA Activated Water. International J of Complementary & Alternative Medicine, 1(1), 00001.
7. Martin, W. J. (2015) Alternative Cellular Energy Pathway Therapy Using KELEA Activated Water. International J Complementary & Alternative Medicine, 2(2), 00051.
8. Martin, W. J. (2015) KELEA, A Natural Energy That Seemingly Reduces Intermolecular Hydrogen Bonding in Water and Other liquids. Open Journal of Biophysics, 5, 69-79.
9. Martin, W. J. and Laurent, D. (2015) Homeopathy as A Misnomer for Activation of the Alternative Cellular Energy Pathway, Evidence for the Therapeutic Benefits of Enercel in a Diverse Range of Clinical Illnesses. International J Complementary & Alternative Medicine, 2(1), 00045
10. Martin, W. J. (2015) Interacting Light Paths Attract KELEA (Kinetic Energy Limiting Electrostatic Attraction) and Can Lead to the Activation of Water. Open Journal of Biophysics, 5, 115-121.
11. Martin, W. J. (2015) Interactive Electric Fields Can Attract KELEA (Kinetic Energy Limiting Electrostatic Attraction) and Can Lead to the Activation of Water. International J Complementary & Alternative Medicine, 1(6), 00034.
12. Martin, W. J. (2016) KELEA (Kinetic Energy Limiting Electrostatic Attraction) May Add to the Measured Weight of an Object. J Modern Physics, 7(6), 461-472.
13. Martin, W. J. (2016) KELEA (Kinetic Energy Limiting Electrostatic Attraction) Offers an Alternative Explanation to Existing Concepts Regarding Wave-Particle Duality, Cold Fusion and Superconductivity. J Modern Physics, 7(15), 1995-2007
14. Martin, W. J. (2016) Preparing and Using KELEA Activated Water to Enhance the Alternative Cellular Energy (ACE) Pathway in the Therapy of Multiple Illnesses. International J Complementary & Alternative Medicine, 3(1), 00059.
15. Martin, W. J. (2015) KELEA Activation of Water and Other Fluids for Health, Agriculture and Industry. Journal of Water Resource and Protection, 7, 1331-1344.
16. Martin, W. J. (2016) KELEA, Cosmic Rays, Cloud Formation and Electromagnetic Radiation, Electropollution as a Possible Explanation for Climate Change. Atmospheric and Climate Sciences, 6(2), 174-179.
17. Martin, W. J. (2016) Cancer as an Insufficiency of Cellular Energy (ICE), Therapeutic Approaches Based on Enhancing the Alternative Cellular Energy (ACE) Pathway. International J Complementary & Alternative Medicine, 3(3), 00074.
18. Martin, W. J. (2016) Insufficiency of Cellular Energy (ICE) in Neurons, From Electrical Hyperactivity to Quiescence. International Journal Complementary Alternative Medicine, 4, 00118.
19. Martin, W. J. (2016) Insufficiency of Cellular Energy (ICE) May Precede Neurodegeneration in Alzheimer's Disease and Be Treatable via the Alternative Cellular Energy (ACE) Pathway. Advances in Alzheimer's Disease, 6(1), 1-12.
20. Martin, W. J. (2016) Insufficiency of Cellular Energy (ICE), The Basis for Many Illnesses Potentially Correctable Using KELEA Activated Water. International J Complementary & Alternative Medicine, 4(1), 00106.
21. Martin, W. J. (2017) Cancer Is Treatable via the Alternative Cellular Energy (ACE) Pathway, Journal of Cancer Therapy, 8, 1279-1290
22. Martin, W. J. (2017) Using KELEA (Kinetic Energy Limiting Electrostatic Attraction) to Improve the Efficiency of Fuel Combustion. Open Journal Air Pollution, 6(3), 103-116.
23. Martin, W. J. (2017) Is KELEA (Kinetic Energy Limiting Electrostatic Attraction) a Source of Chemical Energy? MOJ Biorg Org Chem, 1(2), 54-58.
24. Martin, W. J. (2017) Insufficiency of Cellular Energy (ICE) from the Alternative Cellular Energy (ACE) Pathway Limiting the Specialized Functions of Neuronal Cells. International J Complementary & Alternative Medicine, 4(2), 00112.
25. Martin, W. J. (2017) Hyper-Excitability Followed by Functional Quiescence in Neuronal Cells Caused by Insufficient Cellular Energy (ICE), Treatable by Enhancing the Alternative Cellular Energy (ACE) Pathway. World Journal of Neuroscience, 7(3), 257-266.
26. Martin, W. J. (2017) Tissue Regeneration without Scarring Achieved by Enhancing the Alternative Cellular Energy (ACE) Pathway. Journal of Cosmetics, Dermatological Sciences and Applications, 7(1), 82-98.
27. Martin W J (2018) Is the placebo effect mediated by the Alternative Cellular Energy (ACE) pathway? International J Complementary & Alternative Medicine, 11(4): 231-233.
28. Martin W J (2018) KELEA Activated Water as an Alternative to Stem Cell Injections in Regenerative Medicine. International J Complementary & Alternative Medicine, 11(5), 251-258.
29. Martin W J (2019) Electromagnetic Radiation Causes Weight Loss and Weight Destabilization of Objects with Presumed Elevated Levels of KELEA (Kinetic Energy Limiting Electrostatic Attraction), Relevance to Human Health and to Global Warming. Modern J Physics 10(3), 195-213.

### RELATED PATENT APPLICATIONS BY THE APPLICANT

ACE-pigments and Humic acid as energy sources. Application Ser. No. 10/192,936 Method of assessing and of activating the alternative cellular energy (ACE) pathway in the therapy of diseases. William John Martin Submitted Jan. 16, 2008. Application Ser. No. 12/009,195

Enerceutical mediated activation of the alternative cellular energy (ACE) pathway in the therapy of diseases. Application Ser. No. 12/151,779

Regenerative wound healing using copper-silver citrate composition. Application Ser. No. 12/288,749

Enerceutical activation of the alternative cellular energy (ACE) pathway in therapy of diseases. Application Ser. No. 12/069,597

Method of using the body's alternative cellular energy pigments (ACE-pigments) in the therapy of diseases application Ser. No. 12/378,934

Urine as a source of alternative cellular energy pigments (ACE-pigments) in the assessment and therapy of diseases. Application Ser. No. 12/381,003

Activation of the alternative cellular energy (ACE) pathway in the therapy of diseases. Application Ser. No. 12/802,605

Methods for the detection of alternative cellular energy (ACE) pigments and for monitoring of the ACE pathway in the diagnosis and therapy of diseases. Application Ser. No. 12/802,763

Diagnostic value of systemic ACE pathway activation in the detection by fluorescence of localized pathological lesions. Application Ser. No. 12/804,619

Enerceutical mediated activation of the alternative cellular energy (ACE) pathway in the therapy of diseases. Application Ser. No. 12/151,779

Energy Charged Liquids to Enhance Enerceutical Activation of the Alternative Cellular Energy (ACE) Pathway in the Therapy of Diseases. Application Ser. No. 12/972,344

Energy Charged Alcoholic Beverages for Enhancing the Alternative Cellular Energy Pathway in the Prevention and Therapy of Diseases. Application Ser. No. 12/984,582

Energy Charged Liquids to Enhance Enerceutical Activation of the Alternative Cellular Energy (ACE) Pathway in the Therapy of Diseases application Ser. No. 12/972,344

Methods for Detecting and Monitoring the Activity of Energized Water and Other Liquids Useful for Enhancing the Alternative Cellular Energy Pathway in the Prevention and Therapy of Diseases. Application Ser. No. 13/016,948

Methods for Increasing the Kinetic Activity of Alcohol, Water and Other Liquids, so as to Render the Liquids More Useful in Enhancing the Alternative Cellular Energy Pathway in the Prevention and Therapy of Diseases. Application Ser. No. 13/029,116

Methods for Increasing the Kinetic Activity of Alcohol, Water and Other Liquids, so as to Render the Liquids More Capable of Enhancing the Alternative Cellular Energy Pathway in the Prevention and Therapy of Diseases application Ser. No. 13/040,262

Methods for Increasing the Kinetic Activity of Water and Other Liquids, so as to Render the Liquids More Useful in Enhancing the Alternative Cellular Energy Pathway and in Various Other Agricultural and Industrial Applications. Application Ser. No. 13/166,800

Use of Plants Extracts to Activate Water, Alcohol and Other Liquids. Submitted Oct. 27, 2011. Application Ser. No. 13/272,215.

Methods of Transferring Energies to Water, Alcohols and Minerals. Submitted Nov. 25, 2011. Application Ser. No. 13/304,558.

Use of Certain Foods and Dietary Supplements as Water and Beverage Activating Enerceuticals. Application Ser. No. 14/507,822

Heat as a Method to Enhance the Fluid Activating Ability of Humic Acids, Zeolites and related Enerceuticals. Application Ser. No. 14/294,076

Method of Enhancing the Alternative Cellular Energy Pathway in Humans and Animals Using Wearable Items That Contain KELEA Activated Water. Submitted Feb. 18, 2019. application Ser. No. 16/278,712

Method of Detection and Measurement of a Life Force Energy, Also Known as KELEA. Submitted Feb. 27, 2019. Application number not currently available.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not Applicable

### FIELD OF THE INVENTION

The invention is within the field of a newly defined form of energy, which has major health, agricultural and industrial applications. The energy is referred to by the Applicant as KELEA, being an abbreviation for kinetic energy limiting electrostatic attraction. KELEA is comparable to what traditional oriental medicine has described as the universal life force. Other terms applicable to KELEA include chi, prana, mana, vital energy, orgone, etc. It is also commonly implied in traditional medical teachings that some types of water can provide this type of energy when it is consumed by humans and by animals. The present invention describes a convenient method to detect a deficiency or an adequacy of this energy in humans. The same approach can be applied to measuring the KELEA levels in animals and also plants. As discussed in an earlier patent application, the described method is also finding use in assessing various KELEA amplifying equipment able to establish KELEA enhanced environments. As a relatively easy procedure for measuring KELEA, the present discovery will greatly improve the provision of healthcare to humans and animals.

### BACKGROUND OF THE INVENTION

The human body comprises about 30 trillion cells, which are organized into over 200 different specialized cell types. Individuals are healthy when their cells function normally. To do this, their cells require energy. There are basically three levels of cellular energy requirements. The first is sufficient energy for the cells to stay alive. The second is the additional energy to perform the cells' specialized functions. The third level is the energy needed for cells to respond to adverse events

These third-level responses include the repair of damaged cells and/or the regeneration and replacement of irreversibly damaged and dead cells. The third level of energy is further required by the body to combat infections and to eliminate and to not be overly reactive to toxic materials. Apoptosis or cell suicide should normally occur in cells destined to become a cancer. This cancer-prevention process also requires cellular energy, which may be greater in some cells than the energy required for cellular replication.

Diseases are viewed as an insufficiency of cellular energy at one or more of the three levels. The energy insufficiency can be localized to a particular type of cell or can be more generalized.

While it had been assumed that human and animal cells could only obtain cellular energy from the metabolism of food, the inventor has presented evidence, which disproves this assumption. He proposed that Nature has an energy force, which is attracted to separated electrical charges. The fundamental purpose of this force is presumably to prevent the fusion and elimination of electrostatically attracted opposite electrical charges. He called this force KELEA, as an abbreviation for Kinetic Energy Limiting Electrostatic Attraction.

The inventor further proposed that the fluctuating electrical activities of the brain probably act as the major receiver in humans for this energy. KELEA would then be transferred to the body's fluids as a non-food source of cellular energy. This inventor described this process in numerous research publications and patent applications as the alternative cellular energy (ACE) pathway. Some of this information is embodied in the publications and patent applications, which are incorporated herein by reference.

The inventor further showed that dipolar compounds with separated electrical charges could attract KELEA. Moreover, some of these compounds, as well as certain oscillating electrical devices, can transfer the attracted KELEA to nearby fluids, including water. Within water, KELEA leads to a loosening of the hydrogen bonding between the water molecules. This loosening adds a particular dynamic or kinetic quality to the water, which can be converted to chemical energy.

The exchange of KELEA can also proceed in the reverse direction. That is, placing dipolar compounds next to KELEA activated water can alter the physical properties of the compounds. The absorption of KELEA by non-fluid materials leads to measurable weight increases in the materials. This is in contrast to fluids, such as water and alcohols, in which weight loss predominates, unless the fluid is in a sealed container. The weight loss is due to increased volatility of the activated fluid. This is confirmed in that some closed vials of activated water tested more than 5 years earlier are now half empty, yet the control vials of non-activated water used in the same experiment are still full of water. With non-volatile, dipolar compounds, the attraction of KELEA leads to an increase in weight.

Cellulose is one of the many dipolar compounds that are able to gain weight when stored in the vicinity of KELEA activated water. The cellulose materials included wood chips, cotton fabrics, and ordinary writing paper. Single sheets of 8″×11″ writing paper can be rolled into an 8″ high cylinder using a small strip of adhesive tape. The individual sheets will typically weigh within the range of 4 grams. The weight of a rolled sheet of paper is increased by several milligrams by being placed for some time near a source of activated water. The increased weight will be lost by the subsequent exposure of the paper to electromagnetic radiation, including that emitted by the electronic balance used in the experiments. Data describing these observations are published in the article “Martin W J (2019) Electromagnetic Radiation Causes Weight Loss and Weight Destabilization of Objects with Presumed Elevated Levels of KELEA (Kinetic Energy Limiting Electrostatic Attraction), Relevance to Human Health and to Global Warming. Modern J Physics 10(3), 195-213.” Additional data are included in patent application Ser. No. 16/278,712 and the application submitted on Feb. 27, 2019.

### BRIEF SUMMARY OF THE INVENTION

As described above, it was previously discovered that weight of cellulose containing materials, including regular sheets of paper, cotton fabrics, and pieces of unprocessed wood, will increase if they are stored close to containers of KELEA activated water. Moreover, the weight of the KELEA exposed items will gradually return back to being close to their initial weight during the continuing weighing of the items in an electronic balance. The increase in weight is attributed to the absorption of KELEA by the separated electrical charges on the dipolar cellulose molecules. This is consistent with the premise that KELEA is selectively attracted to separated electrical charges, as are present on dipolar molecules. The additional KELEA increases the measured weight of the molecules. The newly added KELEA can be competitively removed from the molecules by the separated electrical charges evoked by electromagnetic radiation. The addition and subsequent removal of KELEA, as expressed by the reversible increase in weight is especially pronounced when testing paper. This is likely because the cellulose molecules in paper are in the form of shorter, more branched, and interlocking forms allowing for greater exposure of separated electrical charges than in the cellulose molecules in wood. As noted above, the added energy can be subsequently stripped from the cellulose materials by the electromagnetic radiation generated by the electronic balance, or by other devices.

These observations have allowed for a robust assay for the levels of KELEA in water containing mini-hot water bags (pouches). This is measurable as the reversible weight increase by a rolled sheet of regular typing paper placed onto or near to the pouch. Comparable weight gains have occurred over several months, confirming the long-lasting KELEA radiating activity of the water within the pouches. The Applicant envisions that this assay will greatly facilitate the widespread, reliable use of mini-hot water bags as wearable Waterceutical™ pouches by individuals who are intent upon enhancing their level of KELEA.

### BRIEF SUMMARY OF THE INVENTION

As part of these studies, it was noted that even holding of a rolled sheet of paper could lead to measurable changes in the weight of to rolled sheet of paper. Depending upon the occasion, the weight of the rolled sheet of paper could either increase or decrease. It was concluded, therefore, that the change in weight of the paper could be used as a measure of whether the person holding the paper was extracting KELEA from the paper, resulting in a lowered weight, or contributing KELEA to the paper, resulting in an increase in weight of the paper. Various aspects of this basic assay system have been explored over the last several months and are described in this patent application. Among the major developments is the realization that certain forms of conscious mental efforts can also be used in conjunction with the direct use of KELEA activated water to convert an individual from being KELEA extracting to becoming a contributor of KELEA. This discovery has important implications in the provision of improved healthcare for humans and animals.

### BRIEF DESCRIPTION OF THE DRAWINGS

Not Applicable and none included

### DETAILED DESCRIPTION OF THE INVENTION

As described in the earlier patent applications, the weight gain assay is a useful means of measuring the KELEA levels of variously treated water samples. Some of these samples have been added to mini-water bags for placement onto the skin. The bags are regularly checked to confirm their capacity of the water in the bags being able to increase the weight of a sheet of paper laid onto the bag. For this type of testing purpose, the sheet of paper is first exposed to an electromagnetic radiation by being left either in the electronic balance or placed onto a speaker of a radio. This is intended to remove the labile KELEA on the paper. This is shown by the paper no longer showing any further reduction in its weight.

These efforts were extended to see if the weight gain assay could be a measure of the activity of the body's water. Indeed, some earlier experiments confirmed that simply holding onto a roll of paper could increase its weight. What became interesting was that a reverse effect was commonly seen, especially with holding a sheet of paper, which had previously been placed close to activated water. On many occasions, the weight of the paper would show a decline upon holding the paper. In some situations, the reduction in weight upon holding the rolled sheet of paper was also seen with paper that had been exposed to electromagnetic radiation for an extended period of time. When placed into the balance, the weight of the paper would actually increase back towards its earlier level. A more rapid and greater increase occurred when the paper was placed near activated water. Holding the paper once again, led to the same decrease in weight as earlier noted.

It became clear that the direction and extent of induced weight change occurring in a rolled sheet of paper by simply holding the rolled paper for a period of time could potentially provide a measure of the body's KELEA. A series of experiments have supported this utility. These experiments can be summarized as follows:
1. The gains and losses of weight are time dependent up to limits within the range of approximately 45 milligrams (mg) in a typical rolled sheet of paper HO mg per gram or 1%).
2. Holding the rolled sheet of paper using two hands for 30 seconds, with a change in location of the hands at 15 seconds is a suitable period of time to determine the direction of the weight change. The duration of the testing can be extended for several minutes. For animals the rolled paper can be strapped to a body area. The study is best conducted adjacent to or close by to the electronic weighing balance, which should read with a sensitivity of 0.1 mg.
3. The paper used can be obtained from a 500-sheet package stored well away from any activated water and freshly rolled into a column shape for immediate weighing and testing. This helps ensure a lower level of removable KELEA and a more sensitive way of showing an increase in weight upon holding of the paper.
4. Alternatively, a rolled sheet of paper maintained near a source of activated water can be used. The weight of the paper will have been increased by several milligrams by being placed near the activated water. The paper is, therefore, more sensitive to an induced reduction in weight by the withdrawal of KELEA when held by someone with lower KELEA levels. On the other hand, considerably increasing the weight of the paper can present more of a personal challenge in efforts to raise one's KELEA level.
5. As mentioned above, the ability of an individual to withdraw KELEA from a rolled sheet of paper exceeded the reduction in weight that had earlier occurred from the electromagnetic radiation emitted by the electronic balance. Interestingly, when this paper was being reweighed in the electronic balance, its weight increased as if the electromagnetic radiation was now acting as a source of KELEA.
6. It has been possible to convert from withdrawing KELEA from the rolled sheet of paper to contributing KELEA to the same rolled sheet of paper using various means of enhancing the ACE pathway. Typically, a combination of approaches including the use of KELEA activated water and mind-altering mental attitudes, have proven effective within a time period of about 15-30 minutes. These approaches will be detailed below.
7. Major efforts have been undertaken to exclude possible complicating variables, such as time of day or night, humidity, etc., in the readings. Skin areas other than the hands can be used. While direct contact with the skin is more effective, an effect can occur without direct contact but even holding the hands in close vicinity to the rolled sheet of paper.
8. For the inventor, the most predictable correlation with a positive KELEA result is the sense of optimism. Even when a loss of weight is noted, the inventor has now learned to interpret this finding as being a positive transfer of KELEA from the paper to himself. Indeed, the rolled sheet of paper will rather quickly regain some of the lost weight and can be used repeatedly as a source of KELEA. So too can KELEA activated cotton fabrics and wood items be potentially used.
9. The major physical means of changing from a KELEA withdrawing to a KELEA contributing state has been the use of KELEA activated water. Yet, this approach has proven to be especially useful when used in conjunction with adopting a joyful, positive mental attitude. Interestingly, it is as if the use of the KELEA activated water facilitates adopting a sustainable positive mental change leading to an enhanced capacity to transfer KELEA to paper, as evidenced by increasing the weight of the paper.

The above assay has validated the effectiveness of drinking, spraying onto the body and wearing pouches containing activated water, as means of increasing KELEA. For example, when I show a deficiency in KELEA, resulting in a reduced weight of a held rolled sheet of paper, I would typically proceed to drink a 12-fluid ounce bottle of VEW (Vortical Energy Water), available from Starchamber Products, (www.starchamberproducts.com). This product has performed better than my drinking equivalent amounts of MRET (magnetic resonance effect technology) activated water produced by a system developed by Dr. Igor Smirnov. Alternatively, or in addition to drinking the VEW activated water, I would typically spray 1-2 ml of an Enercel Mist onto my face (www.enercel.com). Over time, I would further increase the use of waterceutical pouches, typically made by adding 60 ml of Enercel Max to each of two pouches. The pouches were placed inside my socks against the inner side of my lower legs. These and other pouches would also be used during the night at other skin contact locations. Although, it has occasionally taken up to an hour of dedicated effort, I have invariably been able to convert from reducing the weight of a rolled sheet of paper stored near KELEA activated water to being able to significantly increase its weight. I can now typically achieve the desired result within 15 minutes. I have also challenged myself by outdoing the weight increase in a rolled sheet of paper that had been previously placed onto a garden of flourishing plants.

In performing these studies, I have noted additional benefits from emotionally engaging in aspects of positive psychology. There is seemingly a synergy between exposure to activated water and some of the higher levels of brain functioning. For myself, a sense of optimism and the recalling pleasant autobiographical memories have worked well. Other approaches under investigation include periods of laughing yoga, meditation, companionship, compassion, listening to familiar music, exercise, etc. The described assay procedure should allow individuals to determine their own most effective approach.

Various modifications of the assay can be envisioned, including the use of other cellulose and dipolar compounds containing materials. Various means of discerning increases or decreases in weight are available and much cheaper than a sophisticated electronic balance. For example, a simple fulcrum balance can be constructed for home use. The www.touchscale.co app apparently allows the screen of the attest iphones to measure items to approximately 0.1 gram.

The KELEA monitoring system described in this application or related methods can be used in conjunction with various complementary and alternative medicine (CAM) therapeutic modalities. These include acupuncture, hyperbaric oxygen therapy, homeopathy (activated water), pulsed magnetic therapy, multiwave oscillator devices, Reiki, yoga, etc. This can allow for direct comparisons of the effectiveness of the different modalities in individual patients. The basic approach would be for testing of the individual before and after receiving a treatment. The system can also be an adjunct to the effectiveness of psychiatry and other mainstream medical disciplines, as in empowering the body's KELEA attracting functions. The assay can also be tested for the ability of prayers to enhance the ACE pathway in some individuals.

KELEA enriched cellulose containing materials, including paper and cotton fabrics, can presumably be useful when placed in contact with KELEA deficient individuals. Since the reduction in the weight of such materials will typically be restored by being placed into either a regular environment, or more preferably, a KELEA enhanced environment, the same sheet of paper or cotton fabric can be repeatedly used in an individual. This approach is reminiscent of the use of prayer cloths for healing purposes. As an extension of these studies, both KELEA activated paper and cotton fabric have been shown able to transfer KELEA to water as assessed by reduced surface tension, increased volatility, and more kinetic movements of added neutral red dye particles. The activation could occur even without the need for direct contact of the paper or cotton fabric with the water.

It appears that KELEA may essentially exist on many substances, both liquid and solid, in a stable and in a more labile state. The more labile form can be either naturally released from some dipolar materials, including fluids, possibly because the attracted KELEA induces some oscillating change in the material. This would be similar to the repetitive turning on and off of a KELEA attracting electrical device. The labile form of KELEA can also be competitively exchanged between materials on the basis their relative capacity to attract KELEA. This can explain the bidirectional exchanges of KELEA between different materials depending upon which material presently has the more labile amount of KELEA. The labile KELEA on water molecules is especially significant since it is transferrable between other compounds (chemicals) in the water as chemical energy. Similarly, KELEA can add chemical energy to the hydrocarbons in fuels, including gasoline and diesel.

Humans can also participate in the bidirectional exchange of KELEA with inanimate objects, such as dipolar compounds, including water and cotton fabrics. A major discovery is that the direction of transfer of KELEA can be assessed by whether the weight of the object is increased or decreased. Humans can predictably also transfer KELEA from one to the other, hopefully with the donor being able to regain KELEA through the fluctuating electrical activity of the brain. According to this, it is likely that pets are major source of KELEA to their owners. Certainly, all of the above reasonable regarding humans can apply equally to the health and wellness of animals.

As disclosed in an earlier patent application, the levels of KELEA in various natural and modified environments can also be assessed using the basic principle that KELEA can add to the measured weight of certain materials, including cellulose containing materials.

It is acknowledged that some of the above reasoning is subject to change upon further experimentation. Still the basic discovery applies that KELEA is measurable and transferable.

1. A method for assessing and measuring a type of life force energy, which the Applicant refers to as KELEA, being an abbreviation for kinetic energy limiting electrostatic attraction, in a subject based upon the finding of whether certain cellulose containing and other suitable objects will undergo an increase or a decrease in their weight upon being placed for a period of time in direct contact with, or in close proximity to, the subject; such that an increase in weight of an object provides a quantitative indication of the transfer of KELEA from the subject to the object and a decrease in weight providing a quantitative indication of the transfer of KELEA to the subject from the object, thereby establishing whether the subject has a level of KELEA that is more or less, respectively, than the KELEA level in the object.

2. A method for assessing the ability of a given procedure to increase in a subject, the level of a life force, which the Applicant refers to as KELEA, being an abbreviation for kinetic energy limiting electrostatic attraction, the method comprising the placing of a cellulose containing and other suitable object onto the skin or in close proximity to the skin of the subject for a period of time, such as 30 seconds, prior to and following the completion of the given procedure; in which the finding of a greater relative increase in the weight of the object when the testing is performed after the completion of the procedure provides evidence that the subject has gained in the level of KELEA as a result of the procedure.

3. A method for a subject to receive a life force, which the Applicant refers to as KELEA, being an abbreviation for kinetic energy limiting electrostatic attraction KELEA, comprising the direct contact by the subject with a KELEA activated object, including cellulose containing materials, in which the martial would undergo a loss weight by being in contact with the subject, due to the transfer of KELEA from the material to the subject.

4. The method of claim 1 in which the subject is a human.

5. The method of claim 1 in which the subject is an animal.

6. The method of claim 2 in which the procedure involves the use of one or more medical device that is able to increase the environmental delivery of KELEA to a subject.

7. The method of claim 2 in which the procedure involves the use of KELEA activated water as the means of increasing the delivery of KELEA to a subject.

8. The method of claim 2 in which the procedure involves the use of KELEA activated materials, including the use of cellulose containing materials, as the means of increasing the delivery of KELEA to a subject.

9. The method of claim 2 in which the procedure involves the use of consciousness-related mental exercises and other psychological practices, as the means of increasing the delivery of KELEA to a subject.
